(19) United States
(12) Patent Application Publication
(10) Pub. No.: US 2008/0172087 A1
(43) Pub. Date: Jul. 17, 2008

(54) Anti-slip protection device for a housing head of medical instruments

(76) Inventors: Alexander Von Fuchs, Salzburg (AT); Peter Kratky, Stamberg (DE)

Correspondence Address: KLAUS J. BACH, 4407 TWIN OAKS DRIVE, MURRYSVILLE, PA, 15668, US

(21) Appl. No.: 10/312,629

(22) PCT Filed: Jun. 28, 2001
(86) PCT No.: PCT/EP01/07375

§ 371 Date: Sep. 25, 2003

Related U.S. Application Data
(63) Continuation-in-part of application No. 09/629,740, filed on Jul. 31, 2000, now Pat. No. 6,446,044.

(30) Foreign Application Priority Data
Jun. 28, 2000 (DE) 100 31 436.8

(51) Int. Cl. A61B 17/08

(52) U.S. Cl. 606/213; 606/153

(57) ABSTRACT

The invention relates to an anti-slip protection device for a housing head (21) for medical instruments (20), especially intraluminal instruments (20) for resection or anastomosis operations, comprising a terminal device (2) which can be fixed to the housing head (21) in such a way as to project out and which can be remote-controllably removed from the housing head in at least two sections (3, 4).

### BACKGROUND OF THE INVENTION

The present invention relates to a slide protection device for a housing head of medical instruments, in particular intraluminal instruments for anastomoses following intestinal resections.

Although applicable to any form of intestinal anastomosis, the present invention and the problems on which it is based are described with reference to resection operations in the area of the rectum.

In intestinal resection operations for treating benign or malignant tumors in the area of the descending colon or sigmoid colon or the rectum, the portion of the intestine carrying the tumor is resected, the two disconnected portions of the intestine are brought together and connected by means of a suture, for example a purse-string suture.

This procedure is usually performed using what is called an intraluminal circular clip suture instrument, which can be seen in FIG. 1.

This kind of surgical procedure will be described below with reference to FIGS. 1 through 5. A contact pressure device 27, which has a receiving sleeve 28, is knotted into the proximal portion 30 of the intestine, as is shown in FIG. 2. The corresponding mating piece is introduced through the anus, said mating piece consisting of a housing shaft 22, a grip 23 with a rotation device 24, and of a housing head 21 from which a connection pin 25 can be driven out by means of the rotation device 24 in the direction of the axis of symmetry of the housing shaft 22.

A cutting and clamping mechanism 29 is also provided in the housing head 21 and can be actuated by means of a release lever 34 arranged on the grip 23.

After an operating surgeon has passed the housing shaft 22 with the housing head 21 through the rectum, the connection pin 25 is driven out by means of the rotation device 24, whereupon a tip 26 of the connection pin 25 bores through the center clamped distal route portion 31, as is shown in FIG. 2.

Next, as is shown in FIG. 3, the receiving sleeve 28 of the contact pressure device 27 is engaged with a form fit onto the connection pin 25, with a spring device serving for a secure contact.

Thereafter, as is shown in FIG. 4, the contact pressure device 27 and the housing head 21 are pressed together by means of the contact pin 25 being driven back into the housing head 21 with the aid of the rotation device 24, and, after an indicated position is reached, the cutting and clamping mechanism is released by the operating surgeon pressing the release lever 34.

FIG. 5 shows the result of this surgical procedure, in which in each case an intestinal ring of defined length is cut out from the proximal portion 30 of the intestine and also from the distal portion 31 of the intestine and is removed through the anus together with the intraluminal instrument 20.

The problem on which the present invention is based is that, in the surgical procedure described above, a large number of patients experience postoperative difficulties in voluntarily activating the sphincter muscle. These patients cannot therefore correctly control their bowel movements.

There are principally two reasons for the occurrence of these difficulties. First, the front circumferential edge of the housing head 21, as it is pushed through the intestine, often causes tears and thinning of the musculature of the internal sphincter, and less commonly of the external sphincter.

In addition, as the intraluminal instrument is pushed forward, the soft mucosa of the intestine, that is to say the intestinal mucous membrane, in the inside of the intestinal portion is pushed along by the housing head in the recess of the housing head. The intestinal mucous membrane is thus stretched and is fixed at the anastomosis area. The soft intestinal mucous membrane has a tendency to draw back into the original position. As a result of this stretching tendency, a reduced local perfusion, that is to say an inadequate blood circulation, can occur in the anastomosis area. This insufficient blood supply can lead to anastomosis insufficiency, which represents a serious complication for the patient.

Thinning and tearing of the intestinal serosa can also occur, which also lead to considerable complications.

### SUMMARY OF THE INVENTION

The object of the present invention is therefore to make available a slide protection device for a housing head of medical instruments, which allows these instruments to be introduced and advanced through the intestine without damaging and pushing the intestinal mucous membrane along with it, and which does not impede the subsequent surgical procedure described above.

According to the present invention, the slide protection device consists of a terminal device which can be secured on the housing head in such a way as to project over it and which can be removed from the housing head by remote control in at least two sections.

Compared to the prior art, the slide protection device according to the invention, with the features of claim 1, has the advantage that both the recess and also the circumferential edge of the housing head are covered by the terminal device, as a result of which pushing of the intestinal mucous membrane and damage to the musculature or the intestinal serosa are prevented. In addition, upon reaching the portion of the intestine on which anastomosis is to be performed, the operating surgeon can remove the slide protection device from the housing head by remote control. Thus, the slide protection device does not get in the way of the subsequent procedures, for example the cutting and clamping.

The dependent claims concern advantageous developments and refinements of the slide protection device specified in claim 1.

According to a preferred development, the terminal device is round, in particular skittle-shaped or egg-shaped, with a flattened end at one side. By means of this round shape, the coefficient of friction can be greatly reduced and injuries in the inside of the intestine can be avoided.

According to a further preferred development, the terminal device is made of an elastically deformable material. Thus, on the one hand, there is no risk of injury in the inside of the intestine, and, on the other hand, it can be arranged on housing heads with different diameters, which varies from 25 to 33 mm in the instruments presently available.

According to a further preferred development, the terminal device has, on each of the at least two sections, at least one fastening device for a traction device on which a remote-controlled traction device, in particular a tear-resistant cord, can be arranged. In this way, an inexpensive possibility is afforded of releasing the slide protection device from the housing head by remote control and removing it outside the lumen from the intestine.

According to a further preferred development, the fastening device for a traction device is designed as a recessed fastening eyelet.

According to a further preferred development, the at least two sections are designed symmetrically. This has the advantage that an unnecessarily large section does not have to be withdrawn between the medical instrument and the stretchable intestine.

According to a further preferred development, the at least two sections can be connected to one another via connection devices, in particular predetermined break points designed as plug-in bridges. Thus, the two sections can be connected to one another in an inexpensive and straightforward way and can be easily separated by application of a certain force.

According to a further preferred development, the at least two sections each have flat areas which lie sealingly on one another upon connection of the at least two portions. These areas permit easy application of the force necessary for separation.

According to a further preferred development, each of the at least two sections has, at the flattened end, at least one contact foot which partially encloses to the outer circumference of the housing head. These contact feet serve for additional guiding of the terminal device on the housing head.

According to a further preferred development, the contact feet are designed to be elastically deformable. Thus, even with different housing head diameters, they provide optimum sealing with the outer circumference of the corresponding housing head.

According to a further preferred development, the flattened end of the terminal device has a hollow cylindrical attachment/contact portion. This serves for easy attachment of the terminal device onto a housing head. Since both the whole terminal device and the treatment of the attachment/contact portion are made of an elastically deformable material, simple attachment and securing on housing heads with different diameters is guaranteed.

According to a further preferred development, the attachment/contact portion has a circular contact surface with a conically designed spreader pin guide recess arranged at the center. The conical design facilitates insertion of the connection pin, driven out from the housing head, into the spreader pin guide recess. In this way, the separation of the two sections is permitted by a force application of the driven out connection pin.

In a further embodiment of the invention, the two sections of the slide protection device are designed as rounded cap parts and are connected to one another via releasable traction band fastenings. In this case, the end of the band is preferably secured separately on each cap part, the traction band, between its end fastening and the fastening connecting the caps, being folded at least once approximately at right angles against the inner surface of the cap part. Advantageously, the releasable traction band connection thus affords a releasable connection of the cap parts, which connection is formed by the traction band itself, and the traction band, after being folded twice approximately at right angles against the inner surface of the cap part, is then preferably guided to an end fastening point which lies perpendicular to the line of separation of the cap parts.

If, in addition, it is advantageously provided that the cap part has an edge-side traction band guide formation near its end fastening, it is possible to establish defined traction directions for the removal of each cap part, the guide formation advantageously being incorporated into an edge-side contact formation preferably provided for each cap part for the housing head.

This design permits simple separation of the cap parts and removal after unfolding of the folded together band and thus the release of the connection point between the cap parts.

### DESCRIPTION OF THE FIGURES

Illustrative embodiments of the inventions are shown in the drawings and are explained in more detail in the description below.

FIG. 1 shows a side view of an intraluminal instrument with an unscrewed contact pressure device;

FIGS. 2-5 show perspective views of a resection or anastomosis procedure using the intraluminal instrument from FIG. 1;

FIG. 6 shows a front view of a slide protection device, partly in cross section, according to one illustrative embodiment of the present invention;

FIG. 7 shows a bottom view of the slide protection device from FIG. 6;

FIG. 8 shows a diagrammatic view of a slide protection device arranged on an intraluminal instrument during insertion into a portion of the intestine;

FIG. 9 shows a diagrammatic view of a procedure of separation and removal of a slide protection device from a housing head according to an illustrative embodiment of the present invention;

FIG. 10 shows a further illustrative embodiment of a slide protection device with egg-shaped cap parts and traction bands arranged on a housing head;

FIG. 11 shows an internal view of the slide protection device according to FIG. 10 to illustrate the band fastening at one end and the cap-connecting fastening by the traction bands; and

FIG. 12 shows a side view of the housing head according to FIG. 4 in the first phase after opening of the cap parts.

In the figures, identical reference numbers designate identical components or components with identical function.

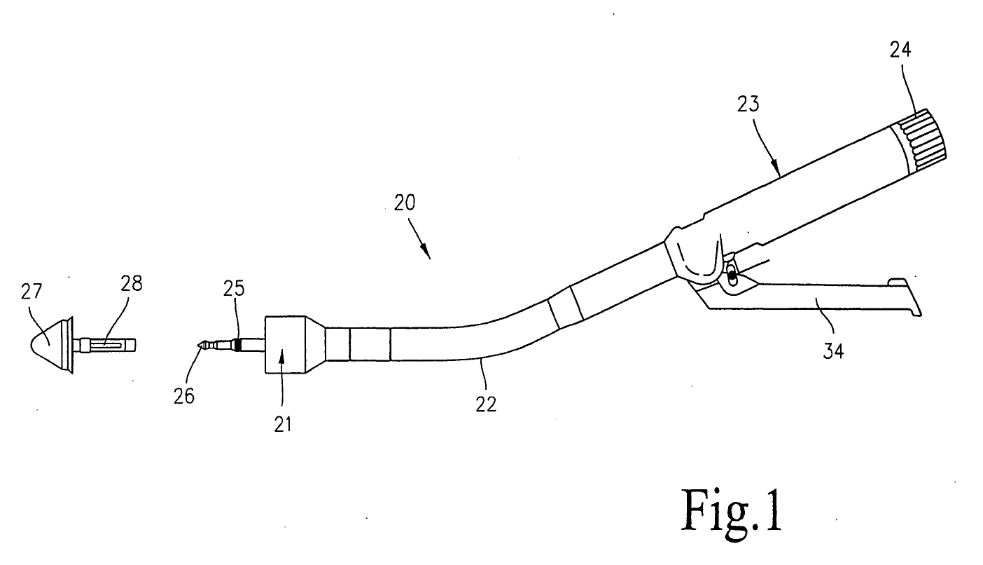

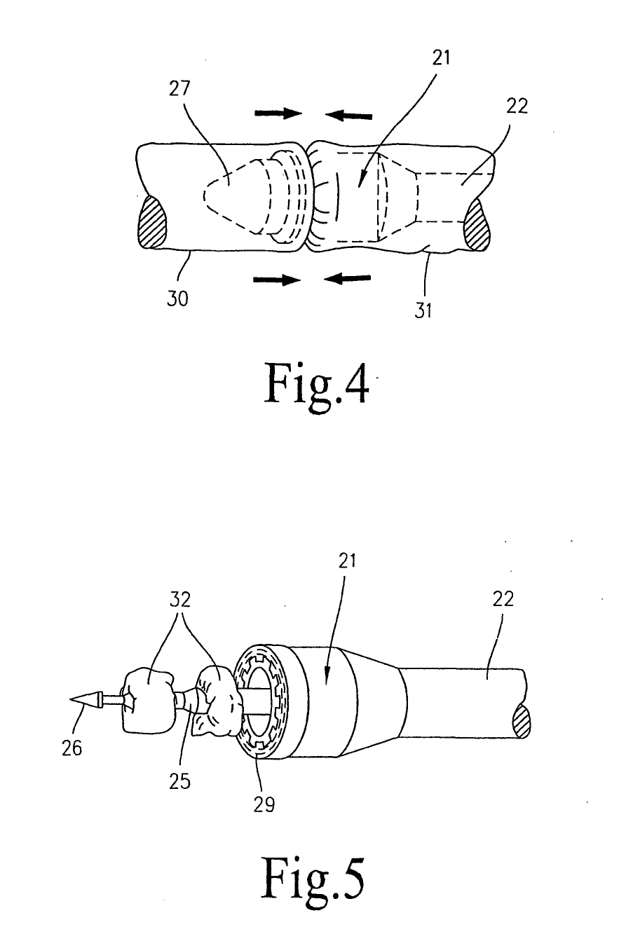

FIGS. 1 through 5 show an intraluminal instrument 20 and the individual steps of a resection operations performed with this instrument. These figures have already been described in detail in the introductory part of the description.

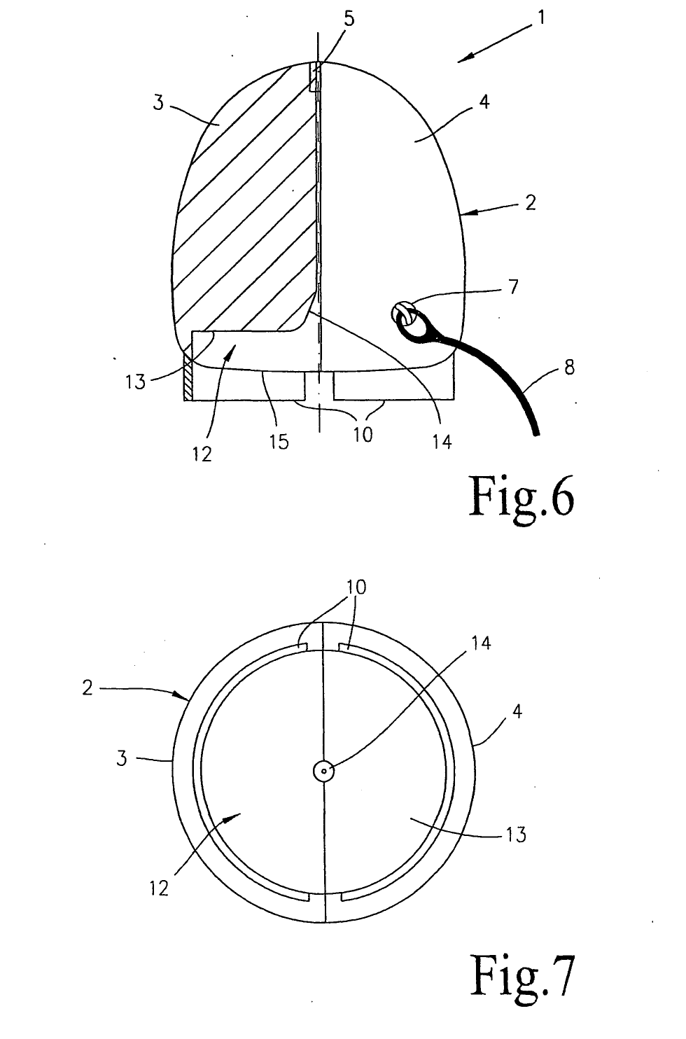

FIG. 6 shows a front view of a slide protection device 1 according to an illustrative embodiment of the present invention, the left half being shown in cross section.

The slide protection device 1 shown in FIG. 1 consists on a terminal device 2 with two sections 3, 4 which at their outer connection line are advantageously connected to one another by means of web-shaped plug-in bridges 5.

According to this illustrative embodiment, the terminal device 2 is egg-shaped with a flattened end 15 at one side. A contact foot 10 in the form of a semi-annular sleeve is formed integrally on this flattened end 15, one for each section 3, 4.

Both the terminal device 2 and the contact feet 10 consist of an elastically deformable material, advantageously of an elastically deformable plastic. The two sections 3, 4 are each made solid in the upper area and touch one another in the state inserted in the terminal device 2 with the plane separating surface.

The terminal device 2 has, in the lower area, as can be seen in the cross-section in FIG. 6 and in the bottom view of the slide protection device 1 in FIG. 7, a hollow cylindrical attachment/contact portion 12 for attachment of the slide protection device 1 onto a housing head 21 of an intraluminal instrument 20.

The attachment/contact portion 12 has a circular contact surface 13 for contact to the end face of the housing head 21. The annular contact surface 13 has a conically tapering spreader pin guide recess 14 at the center.

Since both the terminal device 2 and also the contact feet 10 are made of an elastically deformable material, the terminal device 2 can be arranged on housing heads 21 with different diameters.

Provided on the outside of each of the two sections 3, 4, advantageously in the lower area, there are two fastening devices 7 for a traction band which, in the present illustrative embodiment, are designed as recessed fastening eyelets. A tear-resistant cord 8 is knotted onto each of these fastening eyelets 7.

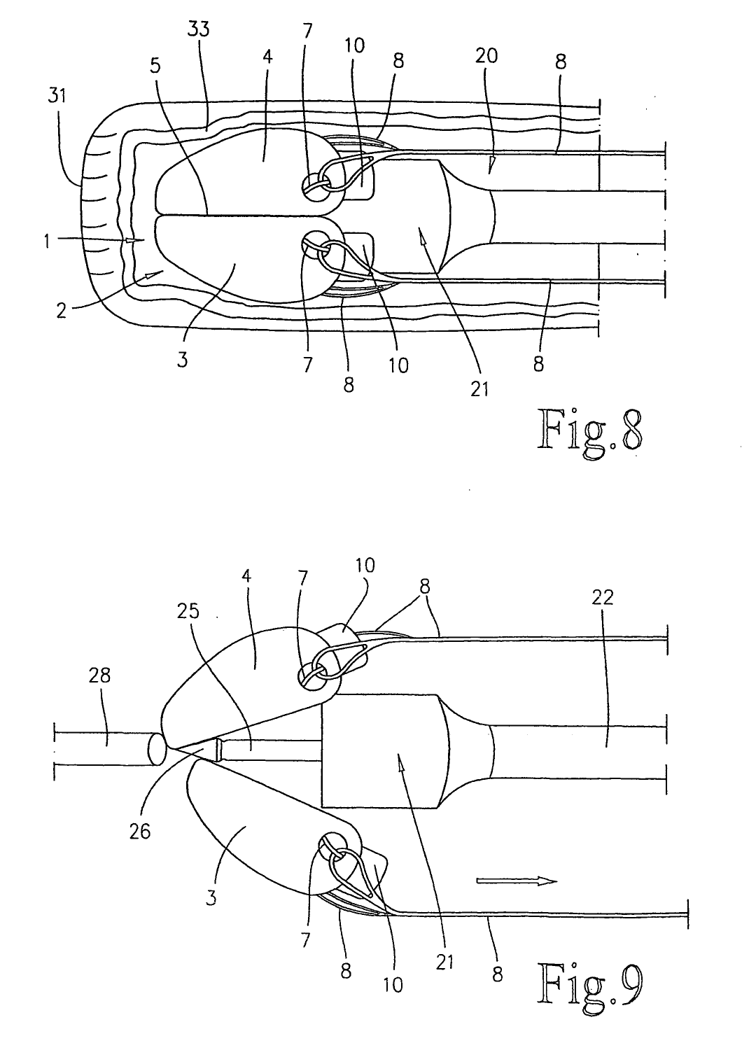

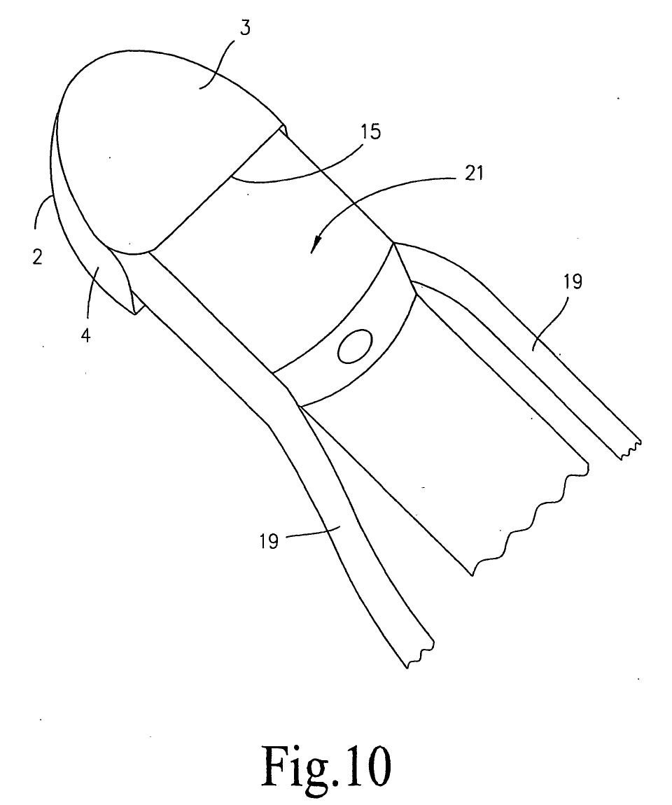

The procedure of a resection or anastomosis operations by means of an intraluminal instrument 20 and an attached housing head 21 attached slide protection device 1 will be described below with reference to FIGS. 8 and 9.

Before insertion of the intraluminal instrument 20, the slide protection device 1 is attached onto the housing head 21 of the instrument 20. Because of the elastic properties with the terminal device 2 and the contact feet 10, this can be done on housing heads 21 with different diameters. The contact surface 13 of the attachment/contact portion 12 lies flat on the end face of the housing head 21. The two contact feet 10 of the terminal device 2 partially enclose the outer circumference of the housing head 21.

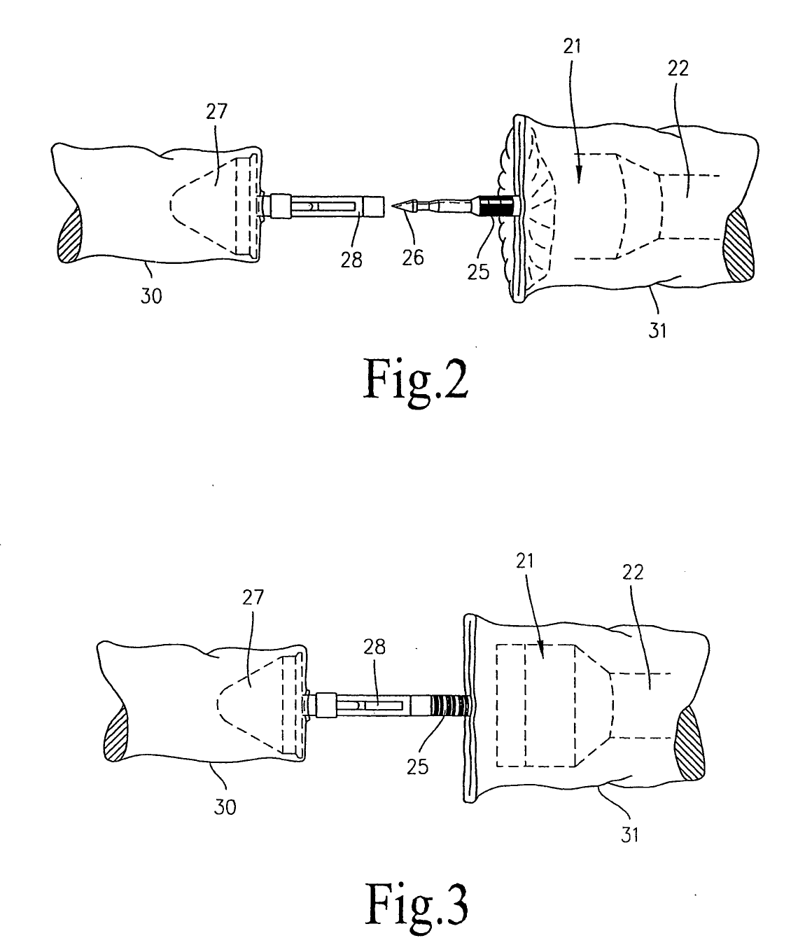

After the contact pressure device 27 of the intraluminal instrument 20 has been passed into the proximal portion 30 of the intestine, as is shown in FIG. 2, the operating surgeon inserts the intraluminal instrument 20 together with housing head 21 into the anus and carefully pushes the instrument 20 forward as far as the intestinal portion where anastomosis is to be performed (generally the rectal stump). On account of the rounded shape of the slide protection device 1, which at the same time serves as a cover for the housing head recess, no mucosal folds are entrained, stretched and thus damaged. In addition, the terminal device 2 is secured so as to project over the housing head 21, as a result of which the sharp edge of the housing head 21 is protected in order to avoid injuries or the like.

When inserting and pushing the instrument 20 forward, the operating surgeon must take care to ensure that the tear-resistant cords 8 at all times hang out of the anus and do not become knotted together in the inside of the intestine.

FIG. 8 shows a diagrammatic view of a housing head 21, with attached slide protection device 1, advanced as far as the portion of the intestine on which re-anastomosis is to be performed. The cords 8 extend from the fastening eyelets 7 in the direction of the distal end of the intestine.

Next, as is shown in FIG. 9, the operating surgeon drives the connection pin 25 out from the inside of the housing head 21 using a rotation device arranged in the grip of the housing. The tip 26 of the connection pin 25 now passes into the spreader pin guide recess 14 in the contact surface 13 of the terminal device 2 and, as it is turned out further, it presses the two sections 3 and 4 apart. The pressure which is thus exerted is chosen to be sufficient to separate the connection by means of the plug-in bridges 5 of the two sections 3 and 4. Thus, when the connection pin 25 is turned out sufficiently far, the slide protection device 1 divides into two equal sections 3 and 4. By pulling the cords 8, it is possible for these two sections 3 and 4 to be removed one after the other without difficulty from the portion of the intestine. Since the intestine is slightly stretchable in the radial direction, the two sections 3 and 4, under a traction force, slip in between the housing head 21 and the inner wall of the portion of the intestine and slide in the direction of the anus. In order to further avoid damage to the intestine or the intestinal mucous membrane in this direction of movement, all the edges, for example of the contact feet 10, are of rounded design.

By removing the slide protection device 1 from the housing head 21 and from the anastomosis portion, the surgical procedure according to the introductory part of the description can be performed in the conventional manner.

According to a second illustrative embodiment of the present invention, the connection device 5 extends along the rotation-symmetrical axis of the terminal device 2. Thus, the extracted connection pin 25 directly separates the predetermined break point which is designed advantageously as a plug-in bridge.

By means of a third illustrative embodiment, the terminal device 2 is not made homogeneously of the same elastically deformable material, but instead as a hollow body which is filled with a suitable in the inert material.

Although the present invention has been described above with reference to preferred illustrative embodiments, it is not limited to these and instead can be modified in numerous ways.

For example, the two sections 3 and 4 can be connected only in a small partial area in an adjoining surface by means of a connection device.

With the present invention, a slide protection device is thus afforded with which medical instruments can be introduced into and advanced through the portions in question without damaging the inside of a hollow organ or the mucous membrane contained therein. In this case, the slide protection device can be removed from the respective housing head from outside by the operating surgeon and can be withdrawn from the portion in question. Thus, the slide protection device does not impede the subsequent important surgical procedure, i.e. the intestinal anastomosis.

In addition, it is an advantage that no plastic parts of the terminal device get into the cutting and clamping mechanism, because the terminate device is arranged outside on the housing head.

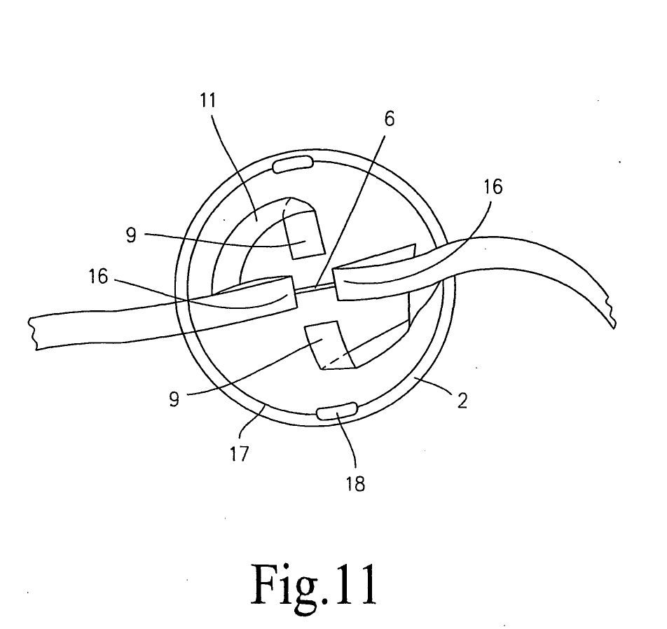

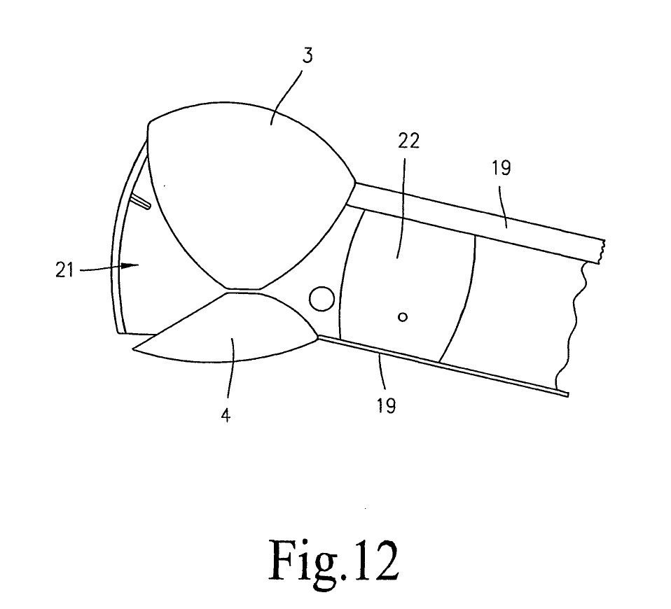

FIG. 10 shows a further illustrative embodiment of a slide protection device according to the invention arranged on a housing head 21. In this slide protection device, two egg-shaped cap parts 3 and 4 are provided which form the terminal device 2. As is indicated in FIG. 11, the two cap parts 3 and 4 are connected by portions of two traction bands 19 at their join 6. The connection is in this case effected by a releasable adhesive, the traction band on the inner surface of the cap parts being folded twice approximately at right angles and finally being fastened securely in the inside of the cap part perpendicular to the join 6. The right-angled band connections are likewise made with a releasable adhesive.

Each cap part 3, 4 has, at the semicircular lower end, a contact edge which in the contact position of the terminal device 2 encloses the outer circumference of the housing head 21. Milled into the inside of each cap part there is a groove 17 with which each cap part bears on the edge of the housing head 21. Offset by 90° with respect to the join 6, each cap part 3, 4 has an edge-side traction band guide formation 18 near its end fastening. The guide formation is designed as a flat groove whose width corresponds approximately to that of the flat traction band 19.

After the terminal device 2 from the state shown in FIG. 11 has been placed in the position according to FIG. 10 on the housing head 21, the traction bands 19 emerge from the egg-cap-shaped terminal device 2 and are guided in the direction of the grip of the medical instrument.

After the described insertion of the instrument 20 through the anal canal into the portion of the rectum on which anastomosis is to be performed, and after the desired position has been reached, each traction band 19 is pulled simultaneously in the direction of the grip, as a result of which the traction band folded together at right angles in the cap inner surface unfolds and slips along the inner surface of the respective cap part in the direction of the flat edge-side guide formation 18. Thus, the easy unfolding of the traction band is ensured by the easy release of the adhesive connection at the join 6 of the two cap parts and by the easy release of the traction band angles fixed with adhesive.

By pulling the traction bands 19 in the direction of the grip, the two cap parts are opened and are guided along the housing head 21 in the direction of the grip. The two cap parts are in this case shaped in such a way that they can slide along the shaft of the inserted instrument and do not constitute an obstacle upon removal of the instrument from the anus. The operating surgeon can now perform the intestinal anastomosis as described.

### LIST OF REFERENCE NUMBERS

1 slide protection device

2 terminal device

3 first section

4 second section

5 web or plug-in bridge

6 join between cap parts/half-shells

7 fastening eyelet

8 traction device

9 secure traction band anchoring of upper pole half-shell

10 contact foot

11 semicircular band guide

12 attachment/contact portion

13 contact surface

14 spreader pin guide recess

15 flattened end

16 folded traction band portion at join 6 releasably bonded

17 contact formation or contact edge half-shell

18 guide formation

19 traction band guide after attachment of closure device

20 intraluminal instrument

21 housing head

22 housing shaft

23 grip

24 rotation device

25 connection pin

26 tip of the connection pin

27 contact pressure device

28 receiving sleeve

29 cutting and clamping mechanism

30 proximal portion of the intestine

31 distal portion of the intestine

32 punched-out ring of tissue

33 mucous membrane

34 release lever

1-17. (canceled)

18. A slide protection device for a housing head of intraluminal instruments for insertion into a body lumen for resection or anastomosis operations, comprising an egg-shaped terminal structure disposed on the housing head of the intraluminal instrument so as to project forwardly from, and cover, the housing head for facilitating sliding of the intraluminal instrument into a body lumen, said terminal structure comprising at least two cover sections joined by releasable connecting means for retaining the slide protection device on the housing head, at least one pulling strip connected to each of the at least two cover sections of the terminal structure, and means for remotely releasing the connecting means so as to separate the at least two sections, said pulling strips extending along the intraluminal instrument for retracting the cover sections, after their release, past the intraluminal instrument from the body lumen.

19. The slide protection device according to claim 18, wherein the egg-shaped terminal structure consists of an elastically deformable material.

20. The slide protection device according to claim 18, wherein each of the at least two cover sections includes at least one fastening structure for the attachment of the pulling strips to the egg-shaped terminal structures.

21. The slide protection device according to claim 20, wherein the fastening structure is a recessed eyelet.

22. The slide protection device according to claim 18, wherein the housing head includes a pin with a conical front end to be driven remotely between the cover sections of the terminal structure for releasing the connecting means and disconnecting the cover sections from one another.

23. The slide protection device according to claim 22, wherein the at least two cover sections are symmetrical.

24. The slide protection device according to claim 23, wherein the at least two cover sections have flat areas adjoining one another when joined in place in front of the housing head and jointly form a conical pin guide recess for directing the front end of the pin between the cover sections in order to cause the release of the connecting means.

25. The slide protection device according to claim 18, wherein the cover sections have flat end faces seating on a front end face of the housing head with contact foot portions extending from the cover sections and jointly surrounding and engaging the housing head.

26. The slide protection device according to claim 25, wherein the contact foot portions are elastically deformable.

27. The slide protection device according to claim 18, wherein the terminal structure comprises two egg-shaped shell sections joined on the inside releasably by adhesive tape sections.

28. The slide protection device according to claim 27, wherein the pull strips are bands firmly connected with their ends to the inside of the shell sections and being provided with adhesive sections forming the adhesive tape sections attached to the shell sections for releasably joining the shell sections.

29. The slide protection device according to claim 28, wherein between the firm attachment locations of the pulling bands to the shell sections and their adhesive sections joining the shell sections, the pull bands are folded at least once approximately at right angles against the inner surface of the respective shell sections.

30. The slide protection device according to claim 29, wherein each shell section is provided at a circumferential area thereof, with a recess sized so as to accommodate the respective pull band extending from within the shell sections.
